(19) United States
(12) Patent Application Publication
(10) Pub. No.: US 2012/0034302 A1
(43) Pub. Date: Feb. 9, 2012

(54) PARTICULATE COMPOSITION AND THE METHOD OF MAKING THE SAME

(76) Inventors: Liangping Yu, Mahwah, NJ (US); Carl Baker, Middletown, NJ (US); Doug DeSisto, SR., Highland Mills, NY (US); Paul Richardson, San Ramon, CA (US)

(21) Appl. No.: 13/148,769

(22) PCT Filed: Feb. 5, 2010
(86) PCT No.: PCT/US10/23271

§ 371 Date: Oct. 20, 2011

Related U.S. Application Data
(60) Provisional application No. 61/151,676, filed on Feb. 11, 2009.

(51) Int. Cl. A61K 9/16; A61K 38/43; A61K 31/522; A61K 31/192; A61K 31/616; A61K 9/20; A61P 3/02; A61K 31/167; A23L 1/05; A23L 1/30; A23L 1/302; A23L 1/304; A23P 1/04; A23P 1/02; B02C 23/00; A61K 36/00

(52) U.S. Cl. 424/465; 424/400; 424/725; 424/94.1; 514/263.34; 514/570; 514/165; 424/489; 514/629; 426/573; 426/578; 426/72; 426/74; 426/61; 426/98; 241/25

(57) ABSTRACT

A particulate material for delivering an active ingredient and a method for making the same is provided. The particulate material can be included in a solid dosage form to provide both high tablet strength and smooth-dissolving mouth-feel. The particulate material includes at least two low moldability sugars, such as, dextrose and sucrose, in an amount of from about 70% to about 100% of the particulate material and is granulated in the presence of a binding agent.

### BACKGROUND OF THE INVENTION

The present invention relates to a particulate composition and its use in a pharmaceutical or nutraceutical solid dosage form, and, in particular, a novel solid dosage form for ease of administration.

Many pharmaceuticals, consumer health products, and supplements come in tablet or capsule dosage forms. It has been reported that more than 35% of the US population has experienced some level of difficulty in swallowing these conventional dosage forms, particularly pediatric and geriatric patients. In order to accommodate these individuals, chewable, rapid dissolve or orally disintegrating tablets have been developed.

Attempts to produce chewable, rapid-dissolving or disintegrating tablets often include compositions of sugar-based excipients, which exhibit high aqueous solubility and sweetness, and are intended to provide smooth mouth-feel and good taste masking. For example, WOWTAB® technology combines low moldability and high moldability saccharides in an attempt to achieve fast dissolving tablets using conventional granulation and tableting techniques. WOWTAB® uses this so-called “smoothmelt action” of sugar and sugar-like (e.g., mannitol) excipients as a drug delivery system. WOWTAB® is based on two U.S. Patents to Mizumoto et al. The first, U.S. Pat. No. 5,576,014 indicates that no single raw material can be used to form tablets having both high strength and fast disintegration properties. Therefore, the '014 patent discloses granulating a saccharide having low moldability with a high moldability saccharide as a binder. The second WOWTAB® patent, U.S. Pat. No. 6,589,554, boasts a quick disintegrating tablet having granules of a sugar and an amorphous sugar. However, after compressing the granules into tablets, the tablets must be humidified and dried to obtain the desired tablet strength. Similarly, PCT publication WO 2004/047810 proposes mannose-based dissolvable tablets which also require humidity treatment.

Other patent applications disclose different compositions intended to obtain rapid dissolving tablets with varying success. PCT publication WO 98/52541 discloses intrabuccally dissolving and partially dissolving compressed tablets formed from low density granules. The low density granulates are made from one or more low density alkali earth metal salts and water soluble carbohydrates—often sugar alcohols. A combination of calcium and sugar alcohols is also disclosed in PCT publication WO 2005/115342, wherein the calcium compound is the active ingredient. Similarly, WO 2006/082499 discloses a melt granulation composition containing a calcium compound and a sugar alcohol. Sugar alcohols, such as those used in the above listed applications, are a known cause of gastric distress.

Accordingly, there is a need for a solid dosage form that exhibits excellent tablet hardness and durability without causing gastric distress or requiring humidity treatment. The solid dosage form should also rapidly dissolve or disintegrate in the mouth, or smoothly melt in the mouth with minimal chewing. The dosage form should be capable of being taken without the need for water. Such a dosage form should provide for ease of administration in order to improve patient compliance. This type of dosage form should be especially beneficial for pediatric and geriatric patients and patients with dysphagia.

In addition, there is a need for a new tablet excipient for swallowable tablets or caplets, where the excipient possesses multi-functional tablet excipient properties.

### SUMMARY

The present invention includes a new particulate material and a method for preparing the particulate material which includes the use of two low-moldability sugars. The invention also includes the method of making a solid dosage form using the particulate material. The low moldability sugars can be present in the particulate material in an amount of from 70% to 100%.

According to the present invention, the particulate is made with at least two low-moldability sugars, such as, and preferably, dextrose and sucrose. The two sugars are granulated together using a binder prepared by solubilizing a minor portion of one of the sugars. Thus, dextrose can be granulated with sucrose using a binder such as, and in a preferred embodiment, solubilized dextrose such that the final dextrose to sucrose weight ratio is from about 85:15 to about 15:85 parts by weight. Additives, such as polysaccharides, in particular, maltodextrin and starch, microcrystalline cellulose, sugar alcohols, etc. can also be used to prepare the particulate. The additives can be up to not greater than about 30% of the resulting particulate material.

In a preferred embodiment of the present invention, the particulate material is formed by granulation of dextrose with sucrose using a weight ratio of from about 70:30 to about 50:50. In a most preferred embodiment of the present invention, the weight ratio of dextrose to sucrose is 70:30 to 60:40. In this present most preferred embodiment, the binder is an aqueous solution of dextrose and maltodextrin, in which about 0.9% of dextrose has been dissolved.

Particulates resulting from the granulation process have a mean diameter size ranging from 50 to 500 microns, and preferably the mean diameter of the particulates is from 100 to 300 microns.

In order to prepare a solid dosage according to the present invention, the particulate material is mixed with one or more active ingredients in a weight ratio of 1:99 to 99:1 and included in a solid dosage form. The preferred ratio between the particulate material and the active ingredient is from 1:9 to 9:1, and more preferred, the ratio is 2:1 to 9:1.

The particulate material described herein provides desirable performance properties in a solid dosage form. In a chewable dosage form, it provides a smooth mouth-feel, good taste, enhanced flowability, excellent content uniformity, sufficient hardness and low friability. For preparation of swallowable tablets or caplets, the particulate material provides good flow, hardness and low friability. The high compactibility of the particulate material leads to excellent structure-forming properties and the particulate material can be directly compressed.

The particulate material of the present invention is also highly beneficial in a multi-particulate powder dosage form, such as sachet and stick pack, to provide rapid and smooth-dissolving mouth-feel, enhanced flowing, and excellent content uniformity.

For a better understanding of the present invention, together with other and further objects, reference is made to the following description, taken in conjunction with the examples, and its scope will be pointed out in the appended claims.

### DETAILED DESCRIPTION

As stated above, the particulate material is a granulate of two low-moldability sugars. Moldability is related to tablet hardness where low moldable sugars tend to have low hardness after compression. Low-moldability sugars in accordance with the invention include, for example, lactose, dextrose, and sucrose. Preferably, the two low-moldability sugars of the invention are dextrose and sucrose.

The dextrose of the invention can be anhydrous or hydrated. Preferably the dextrose is dextrose monohydrate.

The sucrose of the invention can be, for example, powdered sugar or confectioner's sugar.

The percentage of each low-moldability sugar in the particulate material of the invention can be from about 15% to about 85%. For example, a present preferred embodiment includes dextrose and sucrose in an amount of about 70-60% dextrose and about 30-40% sucrose.

The particulate material is formed through granulation using a binding agent. The binding agent of the invention can include any agent capable of forming a particle bridge between the low-moldability sugars during wet granulation. The binding agent can include, for example, sugars, polysaccharides, or polyols, in a solvent. Preferably, the binding agent is a solubilized sugar. Preferably, the solubilized sugar is dextrose.

The particulate material can, optionally, include further additives in an amount of not more than about 30 wt %. These additives can be any additive. Preferable additives include, for example, microcrystalline cellulose, maltodextrin, sugar alcohols and other polysaccharides.

The low-moldability sugars of the invention, and any additives, are granulated together. The granulation process can be performed using methods and equipment known in the industry. Granulation methods that can be used include, for example, single pot, fluid bed top spray granulation, high shear granulation/fluid bed drying combination, continuous fluid bed granulation, fluidized spray drying, pellet production line, and others. Preferably, the granulation method is a top spray fluid bed granulation.

Following binder addition, the particulate material is dried. Preferably the particulate material is dried to a moisture content of not more than about 10%. More preferably, the particulate material is dried to a moisture content of not greater than about 7%.

The resulting particulate material can have a mean particle diameter of 50 to 500 microns. Preferably, the mean particle diameter is 100-300 microns.

The particulate material can be used in conjunction with and for the administration of any active ingredient. For example, the active ingredient can be a pharmaceutical, vitamin, mineral, herb, enzyme, nutritional supplement, or combinations thereof. Preferably, the active ingredient is caffeine, vitamin C, calcium carbonate, magnesium salts, calcium citrate, multivitamins, CoQ10, acetaminophen, ibuprofen, or aspirin. The active ingredient can be a powder or granules. The active ingredients can be coated or non-coated to, for example, provide taste-masking and modified release profiles.

The ratio between active ingredient and particulate material can vary depending on dosage of the active, taste, flow characteristics of the combined materials, and compactibility of the active ingredients. For example, the ratio of particulate material to active ingredient can be from about 1:99 to about 99:1. Preferably, the ratio of particulate material to active ingredient is about 1:9 to about 9:1. Most preferably, the ratio of particulate material to active ingredient is from about 2:1 to about 9:1.

The particulate material and active ingredient can be used to form a solid dosage form. The solid dosage forms can include, for example, tablets, chewable tablets, fast dissolve or orally disintegrating tablets, stick packs, or sachets. The active ingredient can be prepared and included in the solid dosage unit to provide, for example, immediate release, controlled release, modified release, delayed release, or pulsatile release.

Preferably, the solid dosage form is a tablet, caplet, stick pack, or sachet. More preferably, the dosage form is a direct compressed tablet or sachet. Most preferably the dosage is a direct compressed tablet which is a chewable, fast dissolve, or orally disintegrating tablet.

To form a solid dosage form, the particulate material and active ingredient can be mixed and directly compressed into tablets or caplets. In another embodiment, the particulate material and active ingredient can be mixed and directly filled into a powder dosage form such as in stick packs or sachets.

Additional pharmaceutically acceptable excipients can be added to form a solid dosage form. Optional excipients include, but are not limited to, flavoring agents, taste-masking agents, bitter blockers, pH triggers, surfactants, antioxidants, disintegrants, tablet binders, fillers, lubricants, glidants, dispersing agents, and any combinations or mixtures thereof. Preferably, the particulate material and active ingredient(s) form at least 50% of the dosage form, preferably at least 70% and more preferably at least 90% in the final dosage form.

The present invention can be better understood by reference to the following examples. The following examples illustrate the present invention and are not intended to limit the invention or its scope in any manner.

### EXAMPLES

### Example I

### Process for Making Particulate Material

A 4 kg batch of the particulate material was prepared based on the formula shown below.

### Dry Substrate

| | |
|---|---|
| Dextrose monohydrate (Clintose Dextrose) | 2669 g (66.8%) |
| 6X Confectioner's sugar [3% corn starch] (Domino) | 1225 g (30.6%) |
| Microcrystalline cellulose (Avicel PH101) | 58 g (1.4%) |

### Wet Binder

| | | |
|---|---|---|
| Maltodextrin (Maltrin M-100) | 24 | g (0.6%) |
| Dextrose monohydrate (Clintose Dextrose) | 24 | g (0.6%) |
| Purified water (USP) | 472 | g |

A fluid bed granulator was used to prepare the particulate material. The setup and calibration of the fluid bed granulator and top spraying apparatus is understood by those skilled in the art. The binder pump was calibrated to achieve a desired flow rate (e.g. 21 g/min +/−5 g/min). Batch fluidization was started. When proper fluidization and operating temperatures (inlet air target 98° C. +/−4° C., outlet temperature approx 35° C.) were achieved, binder solution application was started at the desired atomization air pressure (e.g. 2.5-3 bar). Upon completion of binder addition, the spraying sequence was stopped and drying sequence was started. The outlet temperature, product temperature, and relative humidity were monitored to determine end point, along with in-process loss on drying (LOD) checks. Upon achieving the desired end point (LOD NMT 10%), the drying process was stopped and the granulated material was discharged into a suitable container. The granulated material was screened through a 20 USSS mesh screen and packaged for further use.

The dry substrate components comprise 98.8% of the formula. The solid binder components comprise 1.2% of the formula. Sufficient USP Purified water is required to obtain a binder solution of approximately 10% solids. The water is driven off during the process and is not part of the finished product.

### Example II

### Compaction of Particulate Material

The particulate material prepared in Example I was compressed into tablets using instrumented Mini Press-II (Globe Pharma) and ½″ flat-faced punches. The tablet formula is shown below.

99% Particulate material

1% Mg-stearate

Total tablet weight: 800 mg

The compaction profile of the particulate material is shown in Table I, below. The particulate material demonstrated excellent compressibility. The tablets that were produced demonstrated good organoleptic properties, especially the smooth dissolve mouth-feel with minimal or no chewing, while maintaining strong mechanical strength. Moreover, the hardness achieved was sufficient to ensure tablet integrity during handling and shipping, while maintaining good mouth-feel.

TABLE I

| Compression | Tablet Weight | Hardness | Friability |
|---|---|---|---|
| (lbs) | (mg) | (kP) | (%) |
| 1000 | 797 | 4.8 | 1.69 |
| 2000 | 800 | 10.9 | 0.45 |
| 3000 | 803 | 16.3 | 0.23 |
| 4000 | 799 | 21.0 | 0.14 |

### Comparative Example III

### Comparison of Granulated Composition and Physically Blended Composition

The dry materials used to prepare the Particulate Material in Example I were physically blended together (67.4% dextrose monohydrate, 30.6% sugar, 1.4% microcrystalline cellulose, and 0.6% maltodextrin). All ingredients were blended in a Turbula blender for five minutes except Mg-stearate, followed by two more minutes blending with 1% Mg-stearate. The blend was compressed into tablets using instrumented Mini Press-II (Globe Pharma) and ½″ flat-faced punches.

The blend did not flow well and lumps formed after blending. It was not possible to maintain a constant tablet weight due to the poor flow properties of the blend. In addition, the tablets produced were not mechanically strong enough for packaging and handling. The structurally sound tablets could not be produced. This blend was not suitable for tablet manufacturing.

### EXAMPLE IV

### Various Particulate Material Compositions

Particulate material was prepared using the process of Example I with varying percentages of each component as shown in Table II, below.

TABLE II

| | Dextrose | 6x Confec- | Microcrystalline | Malto- |
|---|---|---|---|---|
| Formula | Monohydrate | tioner's Sugar | Cellulose | dextrin |
| Formula #1 | 68.8% | 31.2% | 0.0% | 0.0% |
| Formula #2 | 83.5% | 15.0% | 1.2% | 0.3% |
| Formula #3 | 67.7% | 30.8% | 1.2% | 0.3% |
| Formula #4 | 50.7% | 47.8% | 1.2% | 0.3% |
| Formula #5 | 30.5% | 68.0% | 1.2% | 0.3% |
| Formula #6 | 15.4% | 83.1% | 1.2% | 0.3% |

The particulate material prepared above was compressed into tablets using instrumented Mini Press-II (Globe Pharma) and ½″ flat-faced punches. The tablet formula is shown below.

99% Particulate material

1% Mg-stearate

Total tablet weight: 800 mg

Tablet hardness, friability, and compaction ejection force for each formulation are shown in Table III, below.

TABLE III

| | | For- | For- | For- | For- | For- | For- |
|---|---|---|---|---|---|---|---|
| | | mula | mula | mula | mula | mula | mula |
| Compression | Tablet | #1 | #2 | #3 | #4 | #5 | #6 |
| 1000 lbs | Ejection | 8.8 | 4.1 | 5.8 | 12.2 | 10.4 | 11.2 |
| | Force (lbs) | | | | | | |
| | Hardness | 7.6 | 7.7 | 7.2 | 7.5 | 5.0 | 3.9 |
| | (kP) | | | | | | |
| | Friability (%) | 1.6 | 1.1 | 1.6 | 1.7 | 2.6 | 3.4 |
| 2000 lbs | Ejection | 17.2 | 13.2 | 13.6 | 21.9 | 27.6 | 29.6 |
| | Force (lbs) | | | | | | |
| | Hardness | 13.4 | 14.6 | 12.4 | 12.7 | 10.3 | 8.1 |
| | (kP) | | | | | | |
| | Friability (%) | 0.8 | 0.6 | 0.8 | 0.6 | 0.6 | 1.1 |
| 3000 lbs | Ejection | 47.6 | 26.7 | 24.2 | 38.3 | 39.9 | 50.1 |
| | Force (lbs) | | | | | | |
| | Hardness | 21.8 | 23.0 | 17.7 | 19.9 | 16.7 | 14.4 |
| | (kP) | | | | | | |
| | Friability (%) | 0.3 | 0.4 | 0.3 | 0.3 | 0.2 | 0.7 |
| 4000 lbs | Ejection | 61.2 | 36.6 | 36.3 | 47.7 | 50.2 | 67.4 |
| | Force (lbs) | | | | | | |
| | Hardness | 24.1 | 24.0 | 20.7 | 22.9 | 20.5 | 16.4 |
| | (kP) | | | | | | |
| | Friability (%) | 0.2 | 0.2 | 0.2 | 0.2 | 0.2 | 0.5 |

Overall, all formulations granulated and tableted well. All tablets were organoleptically smooth with no grittiness. Tablets either smoothly dissolved in the mouth or dissolved in the mouth with minimal chewing. When the weight ratio of dextrose to sucrose is from 85:15 to 15:85, both sound tablet structure and smooth-dissolving mouth-feel can be achieved.

### Example V

### Vitamin C Tablets

The vitamin C tablets were prepared using the particulate material prepared in Example I. The formula is shown below.

20% Vitamin C (Hebei Welcome Pharmaceutical Co Ltd)

79% Particulate material

1% Mg-stearate

Tablet weight: 800 mg

The blending and tableting conditions were the same as described in Example III. The blends flowed well and showed good compactibility as demonstrated in Table IV, below.

TABLE IV

| Compression | Tablet Weight | Hardness | Friability |
|---|---|---|---|
| (lbs) | (mg) | (kP) | (%) |
| 1000 | 799 | 2.7 | 6.9 |
| 2000 | 800 | 5.5 | 1.5 |
| 3000 | 798 | 8.6 | 0.6 |
| 4000 | 799 | 11.2 | 0.3 |

Both organoleptically and mechanically acceptable tablets were produced when the compression force was above 2,000 lbs.

### Example VI

### Carrying Capacity of Particulate Material

Granulated calcium carbonate (95% calcium carbonate granulated with starch binder, 100-200 microns mean particle size) was used as the active ingredient to demonstrate the carrying capacity of the particulate material prepared in Example I. Granulated calcium carbonate was included in the tablet blends at the levels of 20%, 40%, and 60% of the total tablet weight. The blending and tableting conditions were the same as described in Example III. The compaction profiles of all blends were measured.

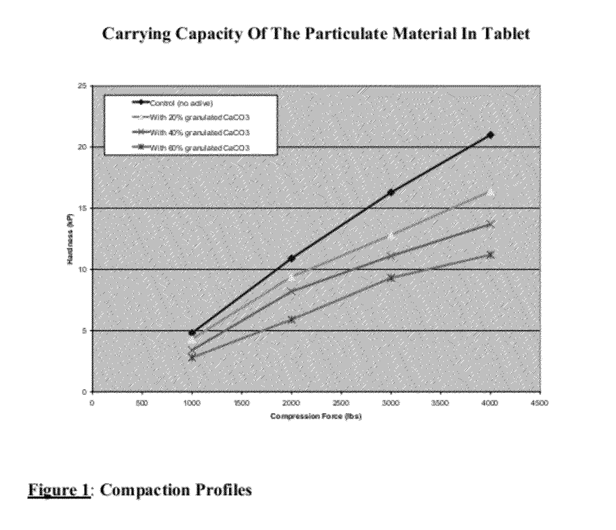

All blends flowed very well and tableted well. The particulate material showed great carrying capability as indicated in FIG. 1. Additionally, the hardness values achieved for each tablet blend were acceptable based on the compression force applied.

### Example VII

### Sustained Release Caffeine in Sachets

The particulate material prepared in Example I was used to deliver sustained release caffeine in sachets. The sustained release caffeine was produced by microencapsulation with a lipid-based coating and sieved through USSS 40 mesh. The formula of the blend is shown below.

25.0% Caffeine encapsulates (67.2% caffeine assay)

74.7% Particulate material

0.3% Berry flavor

Five kg of the above blend was prepared using a MX1-SSJ mixer (Munson Machinery). The blend was filled into sachets using a single lane Sanko packaging machine and a poly/foil/poly film. The targeted sachet weight was 1787 mg to deliver 300 mg caffeine. The blend flowed very well during sachet filling. Excellent content uniformity was achieved for the final dosage unit. The relative standard deviations (RSD) of both sachet weight and caffeine content were below 3%. The bitter taste of the caffeine was also very well masked in the final product. The product dissolved in the mouth rapidly and smoothly, no grittiness or residue particles were detected.

Thus, while there have been described what are presently believed to be the preferred embodiments of the present invention, those skilled in the art will appreciate other and further changes and modifications thereto, and it is intended to include such other changes as come with the scope of the invention as set forth in the following claims.

1. A particulate material for delivering an active ingredient comprising:
at least two low-moldability sugars in an amount of from about 70% to about 100% of the particulate material, and granulated in the presence of a binding agent and having a mean particle size of from 50 to 500 microns.

2. The particulate material according to claim 1 wherein the at least two low-moldability sugars are dextrose and sucrose.

3. The particulate material according to claim 2 wherein the dextrose and sucrose are present in a ratio of 85:15 to 15:85.

4. The particulate material according to claim 3 wherein the dextrose and sucrose are present in a ratio of 70:30 to 50:50.

5. The particulate material according to claim 1 wherein the mean particle size is 100 to 300 microns.

6. The particulate material according to claim 1 wherein the binding agent comprises solubilizied dextrose.

7. The particulate material according to claim 1 wherein the binding agent comprises solubilizied dextrose and maltodextrin.

8. The particulate material according to claim 1 further comprising up to about 30% of an additive selected from the group consisting of maltodextrin, microcrystalline cellulose, starch, and sugar alcohols.

9. A solid dosage form for delivering an active ingredient comprising:
a particulate material having at least two low-moldability sugars in an amount of from about 70% to about 100% of the particulate material, and granulated in the presence of a binding agent and said particulate material having a mean particle size of from 50 to 500 microns and an active ingredient.

10. The solid dosage form according to claim 9 wherein said at least two low-moldability sugars are dextrose and sucrose.

11. The solid dosage form according to claim 10 wherein said dextrose and sucrose are present in a ratio of about 85:15 to about 15:85 in said particulate material.

12. The solid dosage form according to claim 11 wherein said dextrose and sucrose are present in a ratio of about 70:30 to 50:50 in said particulate material.

13. The solid dosage form according to claim 9, wherein said particulate material and said active ingredient are present in a ratio of about 99:1 to about 1:99.

14. The solid dosage form according to claim 13, wherein said particulate material and said active ingredient are present in a ratio of about 9:1 to about 1:9.

15. The solid dosage form according to claim 13, wherein said particulate material and said active ingredient are present in a ratio of about 9:1 to about 2:1.

16. The solid dosage form according to claim 9 wherein said binding agent comprises solubilized dextrose.

17. The solid dosage form according to claim 16 wherein the binding agent further comprises maltodextrin.

18. The solid dosage form according to claim 9 wherein the mean particle size is 100 to 300 microns.

19. The solid dosage form according to claim 9 wherein the active ingredient is selected from the group consisting of pharmaceuticals, vitamins, minerals, herbs, enzymes, nutritional supplements and combinations thereof.

20. The solid dosage form according to claim 9 wherein the active ingredient is selected from the group consisting of caffeine, acetaminophen, ibuprofen, aspirin, and multivitamins.

21. The solid dosage form according to claim 9 wherein the active ingredient is microencapsulated.

22. The solid dosage form according to claim 9, selected from the group consisting of a directly compressed tablet and a directly compressed caplet.

23. The solid dosage form according to claim 9 selected from the group consisting of a chewable tablet, a chewable caplet, a rapid dissolve tablet, a rapid dissolve caplet, an orally disintegrating tablet, and an orally disintegrating caplet.

24. The solid dosage form according to claim 9 is powder dosage form.

25. The solid dosage form according to claim 24, wherein the powder dosage form is selected from the group consisting of a sachet and a stick pack.

26. The solid dosage form according to claim 9 wherein the particulate material and active ingredient together comprise at least 90% of the solid dosage form.

27. The solid dosage form according to claim 26 wherein the particulate material and active ingredient together comprise at least 70% of the solid dosage form.

28. The solid dosage form according to claim 9 wherein the particulate material and active ingredient together comprise at least 50% of the solid dosage form.

29. A method of forming a particulate material comprising:
granulating at least two low-moldability sugars in an amount of from about 70% to about 100% of the particulate material in the presence of a binding agent to form a particulate material and drying said particulate material to a low moisture content.

30. The method according to claim 29 wherein said at least two low-moldability sugars are dextrose and sucrose.

31. The method according to claim 30 wherein said dextrose and sucrose are present in the particulate material in a ratio of 85:15 to 15:85.

32. The method according to claim 31 wherein said dextrose and sucrose are present in the particulate material in a ratio of 70:30 to 50:50.

33. The method according to claim 29 wherein the binding agent comprises solubilized dextrose.

34. The method according to claim 33 wherein the binding agent further comprises maltodextrin.

35. The method according to claim 29 wherein said low moisture content is not more than 10% loss on drying.

36. The method according to claim 35 wherein said low moisture content is not more than 7% loss on drying.

37. The method according to claim 29 wherein said granulating is a wet granulation process.

38. The method according to claim 37 wherein said wet granulation process is a fluid bed granulation.
